(19) United States
(12) Patent Application Publication
(10) Pub. No.: US 2018/0008547 A1
(43) Pub. Date: Jan. 11, 2018

(54) Stable Compositions comprising Linaclotide

(71) Applicants: Shridhar INAGANTI, Hyderabad, Telangana (IN); Venugopala CHOKKASANDRA JAYARAMA REDDY, Hyderabad, Telangana (IN); Jayant KARAJGI, Hyderabad, Telangana (IN); Sivakumaran MEENAKSHISUNDERAM, Hyderabad, Telangana (IN); AUROBINDO PHARMA LTD, Hyderabad, Telangana (IN)

(72) Inventors: Shridhar Inaganti, Hyderabad (IN); Venugopala Chokkasandra Jayarama Reddy, Hyderabad (IN); Jayant Karajgi, Hyderabad (IN); Sivakumaran Meenakshisunderam, Hyderabad (IN)

(73) Assignee: Aurobindo Pharma Ltd, Hyderabad, Telangana (IN)

(21) Appl. No.: 15/548,063

(22) PCT Filed: Feb. 1, 2016
(86) PCT No.: PCT/IB2016/050490

§ 371 Date: Aug. 1, 2017

(30) Foreign Application Priority Data
Feb. 2, 2015 (IN) 499/CHE/2015

(51) Int. Cl. A61K 9/48; A61K 38/10

(52) U.S. Cl. CPC A61K 9/4808; A61K 38/10; A61K 9/485; A61K 9/4858; A61K 9/4866

(57) ABSTRACT

The present invention relates to stable oral composition comprising linaclotide or its pharmaceutically acceptable salts, complexes, polymorphs, hydrates, solvates, enantiomers or racemates, process of preparation thereof and methods of using the same.

### FIELD OF THE INVENTION

The present invention relates to stable oral composition comprising linaclotide or its pharmaceutically acceptable salts, complexes, polymorphs, hydrates, solvates, enantiomers or racemates.

The present invention also relates to process for preparation of stable oral composition comprising linaclotide or its pharmaceutically acceptable salts, complexes, polymorphs, hydrates, solvates, enantiomers or racemates.

The present invention further relates to stable oral composition comprising linaclotide or its pharmaceutically acceptable salts, complexes, polymorphs, hydrates, solvates, enantiomers or racemates for treating Irritable Bowel Syndrome with Constipation (IBS-C) and Chronic Idiopathic Constipation (CIC).

### BACKGROUND OF THE INVENTION

Irritable bowel syndrome (IBS) is a common disorder that affects the large intestine (colon). Irritable bowel syndrome commonly causes cramping, abdominal pain, bloating, gas, diarrhea and constipation. IBS is a chronic condition that you will need to manage long term. Even though signs and symptoms are uncomfortable, IBS unlike ulcerative colitis and Crohn's disease, which are forms of inflammatory bowel disease doesn't cause changes in bowel tissue or increase your risk of colorectal cancer.

Only a small number of people with irritable bowel syndrome have severe signs and symptoms. Some people can control their symptoms by managing diet, lifestyle and stress. Others will need medication and counseling.

Fiber supplements, such as psyllium or methylcellulose, with fluids may help control constipation. Osmotic laxative such as milk of magnesia or polyethylene glycol may be used. Anti-diarrheal medications such as loperamide can help control diarrhea. Some people will benefit from medications called bile acid binders, such as cholestyramine, colestipol or colesevelam Anticholinergics and Antispasmodic medications such as hyoscyamine and dicyclomine can help relieve painful bowel spasms. Tricyclic antidepressants or selective serotonin reuptake inhibitors help relieve depression as well as inhibit the activity of neurons that control the intestines. Some people whose symptoms are due to an overgrowth of bacteria in their intestines may benefit from antibiotic treatment. Some people with symptoms of diarrhea have benefited from rifaximin Two medications are currently approved for irritable bowel syndrome by FDA namely alosetron which is designed to relax the colon and slow the movement of waste through the lower bowel and lubiprostone which works by increasing fluid secretion in the small intestine.

Following patents/patent publications disclose oral compositions comprising linaclotide and process for their preparation.

U.S. Pat. No. 8,802,628 discloses composition comprising linaclotide, Ca²⁺ cation and leucine and process for the preparation of said composition.

US 2009/0253634 discloses dosage unit comprising 30 μg to 1000 μg of linaclotide.

US 2010/0221329 discloses formulation comprising core containing linaclotide dispersed in matrix comprising hydroxypropylmethyl cellulose and polymer surrounding the core.

US 2014/0005128 discloses composition comprising linaclotide, isomalt, cation or pharmaceutically acceptable salt thereof, polyvinyl alcohol and an amine.

US 2014/0018307 discloses composition comprising linaclotide, sterically hindered primary amine, divalent metal cation and formaldehyde scavenger compound.

WO 2013/047795 discloses granular composition obtained by coating a nucleus with a layer containing material having damp-proofing function selected from polyvinyl alcohol, methacrylic acid copolymer S, PVA copolymers, aminoalkyl methacrylate copolymer E, ethylcellulose and methacrylic acid copolymer LD followed by linaclotide layer and then layer containing material having damp-proofing function.

WO2015/089335 discloses a pharmaceutical composition comprising linaclotide; Ca²⁺; histidine; and polyvinyl alcohol, wherein the molar ratio of Ca²⁺; histidine: linaclotide is between 30-80:80-120:1.

WO2015/089326 discloses a delayed release pharmaceutical compositions comprising linaclotide or pharmaceutically acceptable salts thereof, as well as to various methods and processes for the preparation and use of the compositions.

The prior arts discussed hereinbefore disclose various compositions comprising linaclotide and process for their preparation. However, the inventors of the present invention have endeavored to develop stable compositions comprising linaclotide with enhanced solubility and bioavailability. Further, the compositions of the present invention are safe to use and provide the desired drug release both in-vivo and in-vitro for the intended duration.

### Objective of the Invention

The objective of the present invention is to provide stable oral composition comprising linaclotide or its pharmaceutically acceptable salts, complexes, hydrates, solvates, enantiomers or racemates and one or more pharmaceutically acceptable excipients.

Another objective of the present invention is to provide process for preparation of stable oral composition comprising linaclotide or its pharmaceutically acceptable salts, complexes, hydrates, solvates, enantiomers or racemates and one or more pharmaceutically acceptable excipients.

Another objective of the present invention is to provide stable oral composition for treating irritable bowel syndrome with constipation and chronic idiopathic constipation using the oral composition comprising linaclotide or its pharmaceutically acceptable salts, complexes, hydrates, solvates, enantiomers or racemates and one or more pharmaceutically acceptable excipients.

Yet another objective of the present invention is to provide stable oral composition comprising linaclotide or its pharmaceutically acceptable salts, complexes, hydrates, solvates, enantiomers or racemates, which exhibit comparative in-vitro dissolution profile with respect to the reference product LINZESS® and which exhibit comparative in-vivo bioequivalence with respect to the reference product LINZESS®.

### SUMMARY OF THE INVENTION

In an embodiment, the present invention relates to stable oral composition comprising linaclotide or its pharmaceutically acceptable salts, complexes, hydrates, solvates, enantiomers or racemates and one or more pharmaceutically acceptable excipients.

In another embodiment, the present invention relates to process for preparation of stable oral composition comprising linaclotide or its pharmaceutically acceptable salts, complexes, hydrates, solvates, enantiomers or racemates and one or more pharmaceutically acceptable excipients.

In another embodiment, the present invention relates to a stable oral composition comprising linaclotide, wherein said composition is substantially free of primary amine and/or cation.

In an embodiment, the present invention relates to stable oral composition comprising linaclotide or its pharmaceutically acceptable salts, complexes, hydrates, solvates, enantiomers or racemates, and one or more pharmaceutically acceptable excipients selected from a group comprising binder, cation, lubricant, polymer and glidant, wherein said composition is substantially free of primary amine.

In an embodiment, the present invention relates to stable oral composition comprising linaclotide or its pharmaceutically acceptable salts, complexes, hydrates, solvates, enantiomers or racemates, and one or more pharmaceutically acceptable excipients selected from a group comprising binder, primary amine, lubricant, polymer and glidant, wherein said composition is substantially free of cation.

In an embodiment, the present invention relates to stable oral composition comprising linaclotide or its pharmaceutically acceptable salts, complexes, hydrates, solvates, enantiomers or racemates, and one or more pharmaceutically acceptable excipients selected from a group comprising binder, secondary amine, cation, lubricant, polymer and glidant.

In another embodiment, the present invention relates to process for preparation of stable oral composition comprising linaclotide comprising:

i) layering linaclotide over a core to form pellets;
ii) lubricating the drug coated pellets; and
iii) encapsulation.

In another embodiment, the present invention relates to process for preparation of stable oral composition comprising linaclotide comprising:

i) layering linaclotide over a core to form pellets;
ii) optionally seal coating the pellets;
iii) lubricating the pellets; and
iv) encapsulation.

Another aspect of the present invention is to provide method of using the stable oral composition comprising linaclotide which comprises administration of an effective amount of said composition to a subject in need thereof.

In another embodiment, the present invention relates to stable oral composition comprising linaclotide for treating irritable bowel syndrome predominant constipation and chronic idiopathic constipation.

### DETAILED DESCRIPTION OF THE INVENTION

In an embodiment, the present invention relates to a stable oral composition comprising linaclotide or its pharmaceutically acceptable salts, complexes, hydrates, solvates, enantiomers or racemates and one or more pharmaceutically acceptable excipients.

In another embodiment, the present invention relates to process for preparation of a stable oral composition comprising linaclotide or its pharmaceutically acceptable salts, complexes, hydrates, solvates, enantiomers or racemates and one or more pharmaceutically acceptable excipients.

In another embodiment, the present invention relates to a stable oral composition comprising linaclotide, wherein said composition is substantially free of primary amine and/or cation.

In an embodiment, the present invention relates to a stable oral composition comprising linaclotide or its pharmaceutically acceptable salts, complexes, hydrates, solvates, enantiomers or racemates, and one or more pharmaceutically acceptable excipients selected from the group comprising binder, cation, lubricant, polymer and glidant, wherein said composition is substantially free of primary amine.

In an embodiment, the present invention relates to a stable oral composition comprising linaclotide or its pharmaceutically acceptable salts, complexes, hydrates, solvates, enantiomers or racemates, and one or more pharmaceutically acceptable excipients selected from the group comprising binder, primary amine, lubricant, polymer and glidant, wherein said composition is substantially free of cation.

In an embodiment, the present invention relates to stable oral composition comprising linaclotide or its pharmaceutically acceptable salts, complexes, hydrates, solvates, enantiomers or racemates, a secondary amine, and one or more pharmaceutically acceptable excipients selected from the group comprising binder, cation, lubricant, polymer and glidant.

In another embodiment, the present invention relates to process for preparation of a stable oral composition comprising linaclotide comprising:

i) layering linaclotide over a core to form pellets;
ii) lubricating the coated pellets; and
iii) encapsulation.

In another embodiment, the present invention relates to process for preparation of a stable oral composition comprising linaclotide comprising:

i) layering linaclotide over a core to from pellets;
ii) optionally seal coating the pellets;
iii) lubricating the pellets; and
iv) encapsulation.

Another aspect of the present invention is to provide method of using a stable oral composition comprising linaclotide which comprises administration of an effective amount of said composition to a subject in need thereof.

In an embodiment, the primary amine is an amino acid. In yet another embodiment, the amino acid is a naturally-occurring amino acid selected from a group comprising phenylalanine, alanine, glutamic acid, aspartic acid, glutamine, leucine, methionine, asparagine, tyrosine, threonine, isoleucine, tryptophan, methionine and valine. The amount of primary amine may range from about 0.2% to about 4% by weight of the composition.

In an embodiment, the secondary amine is an amino acid which is L-proline. The amount of secondary amine may range from about 0.2% to about 6% by weight of the composition.

In an embodiment, the present invention provides stable oral composition comprising linaclotide or its pharmaceutically acceptable salts, complexes, hydrates, solvates, enantiomers or racemates, in the form of coated or uncoated granules, tablets, mini tablets, coated or uncoated beadlets or coated or uncoated pellets filled in to hard gelatin capsules by conventional techniques.

In another embodiment, the present invention provides process for preparation of stable oral composition comprising linaclotide comprising layering solution or dispersion or suspension of linaclotide over the inert core.

In another embodiment, the present invention provides process for preparation of stable oral composition comprising linaclotide in fluid bed processor by spraying drug solution or dispersion or suspension of linaclotide over the inert core.

In an embodiment, the present invention provides stable oral composition comprising linaclotide or its pharmaceutically acceptable salts, complexes, hydrates, solvates, enantiomers or racemates, in the range of about 80 microgram to about 350 microgram, preferably in the range of about 100 microgram to about 320 microgram and more preferably in the range of 120 microgram to 290 microgram.

In another embodiment, the present invention provides optional seal coating layer over linaclotide coated core.

In another embodiment, the present invention provides seal coating solution comprising polymer dispersion selected from a group comprising hydroxypropylmethyl cellulose or hydroxypropyl cellulose optionally with one or more excipients selected form the group comprising plasticizers, glidants and diluents.

In another embodiment, the present invention relates to process for preparation of stable oral composition comprising linaclotide comprising:

i) loading the inert cores in fluid bed processor,
ii) preparing the aqueous solution comprising cation, hydrochloric acid, hydroxypropylmethyl cellulose and linaclotide;
iii) coating drug solution of step (ii) over the cores of step (i) to form drug layered pellets;
iv) optionally seal coating the drug layered pellets using polymer dispersion along with one or more excipients;
v) lubricating the pellets; and
vi) encapsulating in capsules.

In another embodiment, the present invention relates to process for preparation of stable oral composition comprising linaclotide comprising:

i) loading the inert cores in fluid bed processor,
ii) preparing the aqueous solution comprising primary amine, hydrochloric acid, hydroxypropylmethyl cellulose and linaclotide;
iii) coating drug solution of step (ii) over the cores of step (i) to form drug layered pellets;
iv) optionally seal coating the drug layered pellets using polymer dispersion along with one or more excipients;
v) lubricating the pellets; and
vi) encapsulating in capsules.

In another embodiment, the present invention relates to process for preparation of stable oral composition comprising linaclotide comprising:

i) loading the inert cores in fluid bed processor,
ii) preparing the aqueous solution comprising cation, secondary amine, hydrochloric acid, hydroxypropylmethyl cellulose and linaclotide;
iii) coating drug solution of step (ii) over the cores of step (i) to form drug layered pellets;
iv) optionally seal coating the drug layered pellets using polymer dispersion along with one or more excipients;
v) lubricating the pellets; and
vi) encapsulating in capsules.

In an embodiment, the linaclotide layering over inert cores and seal coating are carried out in fluid bed processor at a temperature of 30±5° C.

In another embodiment, the present invention relates to method of using the stable oral composition comprising linaclotide for treating irritable bowel syndrome predominant constipation and chronic idiopathic constipation.

In another embodiment, the present invention provides the use of stable oral composition comprising linaclotide for the treatment of irritable bowel syndrome predominant constipation and chronic idiopathic constipation.

In another embodiment, the present invention provides a method of treating a patient with irritable bowel syndrome predominant constipation and/or chronic idiopathic constipation, comprising administering a stable oral composition comprising linaclotide, wherein said composition is substantially free of primary amine and/or cation.

The term “composition” or “formulation” or “dosage form” or “medicinal preparation” as used herein synonymously include solid dosage forms such as granules, multiunit particulate systems (MUPS), pellets, spheres, tablets, capsules, mini-tablets, beads, crystals, particles and the like meant for oral administration.

Further, as used herein the term inert core refers to a pharmaceutically acceptable core for use in pharmaceutical formulations which is inert. Exemplary cores include inert spheroids, Nonpareils, sugar spheroids, Cellets®, Celphere®, microcrystalline cellulose spheres, spheres made of microcrystalline cellulose and one or more sugars, such as lactose, and combinations comprising one or more of the foregoing cores.

As used in this specification, the singular forms “a”, “an” and “the” include plural references unless the context clearly dictates otherwise. Thus for example, a reference to “a process” includes one or more processes, and/or steps of the type described herein and/or which will become apparent to those persons skilled in the art.

The term “pharmaceutical acceptable excipient” as used herein refers to additives useful for converting pharmacologically active compounds into pharmaceutical dosage forms which are suitable for administration to patients. Suitable excipients include diluents, binders, matrix forming agents, lubricants, glidants, film forming polymers, plasticizers and coloring agents. Other pharmaceutically acceptable excipients can also be included.

Suitable diluents used according to the present invention include but are not limited to water soluble or water insoluble diluents comprising sucrose, dextrose, lactose, mannitol, sorbitol, starch, microcrystalline cellulose, silicified microcrystalline cellulose, calcium silicate and the like or combination thereof. The amount of diluent may range from about 5% to 95% by weight of the composition.

Binders refer to polymers which provide binding effect. The binders according to the present invention include but are not limited to ethyl cellulose, hydroxypropyl cellulose, hydroxypropyl methylcellulose, methylcellulose and hydroxyethyl cellulose, carboxymethyl cellulose; starch and its derivatives; pregelatinized starch, hydrocolloids; sugars; polyvinyl pyrrolidone, copovidone, polyethylene glycol; sugar esters such as sucrose stearate, sucrose palmitate or sucrose laurate or glyceryl behenate and the like. The amount of binder may range from 0% to about 20% by weight of the composition.

Suitable lubricants used according to the present invention include but are not limited to magnesium stearate, hydrogenated castor oil, calcium stearate, sodium stearyl fumarate, talc, vegetable oils, stearic acid, fumaric acid and the like. The amount of lubricants may range from about 0.1% to about 6% by weight of composition.

The inclusion of a plasticizer in the polymer dispersion improves the physical properties of the film. Suitable plasticizers according to the present invention include but are not limited to polyethylene glycol, propylene glycol, diethyl phthalate, castor oil, triethyl citrate, tributyl citrate and dibutyl sebacate. The amount of plasticizer may range from about 5% to 30% by weight of the composition.

Suitable glidants according to the present invention include but are not limited to talc, colloidal silicon dioxide, dibasic calcium phosphate, tribasic calcium phosphate and pregelatinized starch. The amount of glidant may range from about 0.1% to 8% by weight of the composition.

Suitable cations according to the present invention include but are not limited to Mg²⁺, Ca²⁺, Zn²⁺, Mn²⁺, Na⁺ or Al³⁺. In some embodiments, the cation is provided as, without limitation, magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate. In further embodiments, the cation is provided as magnesium chloride, calcium chloride, calcium phosphate, calcium sulfate, zinc acetate, manganese chloride, potassium chloride, sodium chloride or aluminum chloride. The amount of cation may range from about 0.5% to 5% by weight of the composition. As used herein, the term “substantially free of primary amine” means primary amine if present is contained in an amount less than about 1% based on total weight of the composition.

As used herein, the term “substantially free of cation” means cation if present is contained in an amount less than about 1.0% based on total weight of the composition.

As used herein, the term “stable” means less than 1% of known and/or unknown impurities and less than 5% of total impurities.

Some of the known impurities for linaclotide are: “oxidation product”, “Formaldehyde imine product” and “N-Acetyl Linaclotide”.

| Impurity | Structure |
|---|---|
| Linaclotide degradation/impurity (Oxidation product) | 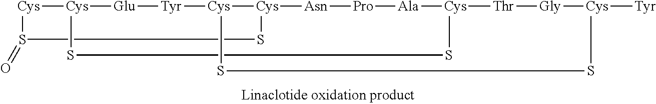 |
| Linaclotide degradation/ impurity (hydrolysis product) | 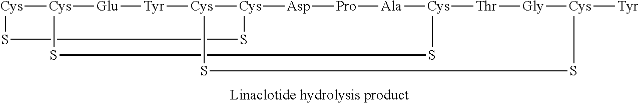 |
| Linaclotide degradation/impurity (formaldehyde imine product) | 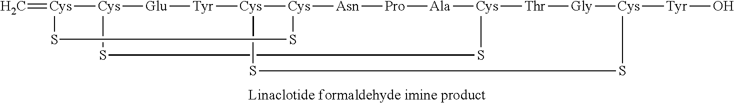 |
| Linaclotide inactive | Cys-Cys-Glu-Tyr (CCEY) |
| degradation product | |
| Linaclotide inactive | Cys-Cys-Asn-Pro-Ala-Cys-Thr-Gly-Cys-Tyr |
| degradation product | (CCNPACTGCY) |
| Linaclotide impurity | 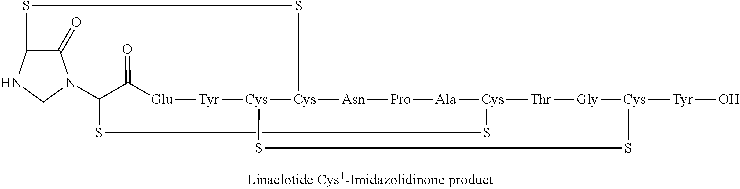 |

The following examples further exemplify the invention and are not intended to limit the scope of the invention in any manner whatsoever. It is obvious to those skilled in the art to find out the composition for other dosage forms and substitute the equivalent excipients as described in this specification or with the one known to the industry.

### Example 1

| | Quantity (mg/capsule) | | Quantity (mg/capsule) | |
|---|---|---|---|---|
| Ingredients | 145 mcg | 290 mcg | 145 mcg | 290 mcg |
| MCC Spheres | 54.5 | 109 | 54.5 | 109 |
| Drug loading | | | | |
| Linaclotide | 0.145 | 0.290 | 0.145 | 0.290 |
| Hydroxy propyl methyl | 0.407 | 0.813 | 0.560 | 0.950 |
| cellulose 5 cps | | | | |
| Calcium chloride | 0.975 | 1.95 | 1.25 | 2.30 |
| dihydrate | | | | |
| Hydrochloric acid | Qs | Qs | Qs | Qs |
| Purified water | Qs | Qs | Qs | Qs |
| Drug loaded pellets | 56.027 | 112.053 | 56.455 | 112.540 |
| weight | | | | |
| Seal coating | | | | |
| Hydroxy propyl methyl | 5.603 | 11.205 | 6.50 | 12.250 |
| cellulose 3 cps | | | | |
| Hydrochloric acid | Qs | Qs | Qs | Qs |
| Purified water | Qs | Qs | Qs | Qs |
| Seal coated pellets | 61.630 | 123.258 | 62.955 | 124.790 |
| weight | | | | |
| Lubrication | | | | |
| Talc | 0.616 | 1.232 | 0.850 | 1.450 |
| Total pellets weight | 62.246 | 124.490 | 63.805 | 126.240 |
| Encapsulation | | | | |
| Hard Gelatin Capsule | Size 3 | Size 2 | Size 3 | Size 2 |
| Shells | | | | |

Qs—quantity sufficient

The process involved in manufacturing composition as given in Example 1 comprises the following steps:

### Drug Layering:

(i) Hydrochloric acid was added to water to get a solution of pH between 3 and 4.
(ii) Calcium chloride dihydrate was added to solution of step (i) under stirring to get clear solution.
(iii) Hydroxy propyl methyl cellulose was added to solution of step (ii) and continue stirring till clear solution is obtained.
(iv) The pH of solution of step (iii) was measured and adjusted with hydrochloric acid to between 3 and 4.5.
(v) The solution of step (iv) was kept in an ice bath to attain temperature between 2 and 8° C. with nitrogen gas bubbling.
(vi) Linaclotide was added to solution of step (v) under stirring.
(vii) Microcrystalline cellulose beads were loaded in fluid bed coater and heated prior to drug layering.
(viii) The drug coating solution of step (vi) was sprayed on to the beads of step (vii) and dried.

### Seal Coating (Optional):

(ix) Hydrochloric acid was added to water to get a solution of pH between 3 and 4.
(x) Hydroxypropylmethyl cellulose 3 cps was added to the solution of step (ix) and stirring was continued until clear solution was obtained.
(xi) Drug layered pellets were loaded in fluid bed coater followed by spraying of solution of step (x) and drying.
(xii) The pellets of step (xi) were lubricated with sifted talc and filled in capsules.

### Example 2

| | Quantity (mg/capsule) | | Quantity (mg/capsule) | |
|---|---|---|---|---|
| Ingredients | 145 mcg | 290 mcg | 145 mcg | 290 mcg |
| MCC Spheres | 54.5 | 109 | 54.5 | 109 |
| Drug loading | | | | |
| Linaclotide | 0.145 | 0.290 | 0.145 | 0.290 |
| Hydroxy propyl methyl | 0.407 | 0.813 | 0.450 | 1.050 |
| cellulose | | | | |
| Leucine | 0.422 | 0.845 | 0.400 | 0.750 |
| Hydrochloric acid | Qs | Qs | Qs | Qs |
| Purified water | Qs | Qs | Qs | Qs |
| Drug loaded pellets | 55.474 | 110.948 | 55.495 | 111.090 |
| weight | | | | |
| Seal coating | | | | |
| Hydroxy propyl methyl | 5.547 | 11.094 | 5.250 | 10.275 |
| cellulose | | | | |
| Hydrochloric acid | Qs | Qs | Qs | Qs |
| Purified water | Qs | Qs | Qs | Qs |
| Seal coated pellets | 61.021 | 122.042 | 60.745 | 121.365 |
| weight | | | | |
| Lubrication | | | | |
| Talc | 0.612 | 1.220 | 0.820 | 1.540 |
| Total pellets | 61.633 | 123.262 | 61.565 | 122.905 |
| weight | | | | |
| Encapsulation | | | | |
| Hard Gelatin Capsule | Size 3 | Size 2 | Size 3 | Size 2 |
| Shells | | | | |

Qs—quantity sufficient

The process involved in manufacturing composition as given in Example 2 comprises the following steps:

### Drug Layering:

(i) Hydrochloric acid was added to water to get a solution of pH between 3 and 4.
(ii) Leucine was added to solution of step (i) under stirring to get clear solution.
(iii) Hydroxy propyl methyl cellulose was added to solution of step (ii) and stirring was continued till clear solution is obtained.
(iv) The pH of solution of step (iii) was measured and adjusted with hydrochloric acid to between 3 and 4.5.
(v) Solution of step (iv) was kept in ice bath to attain temperature between 2 and 8° C.
(vi) Linaclotide was added to solution of step (v) under stirring.
(vii) Microcrystalline cellulose beads were loaded in fluid bed coater and heated prior to drug layering.
(viii) The drug coating solution of step (vi) was sprayed on to the beads of step (vii) and dried.

### Seal Coating (Optional):

(ix) Hydrochloric acid was added to water to get a solution of pH between 2.5 and 4.5.
(x) Hydroxypropylmethyl cellulose was added to the solution of step (ix) under stirring.
(xi) Drug layered pellets were loaded in fluid bed coater followed by spraying of solution of step (x) and drying.
(xii) The pellets of step (xi) were lubricated with sifted talc and filled in capsules.

### Example 3

| | Quantity | | Quantity | |
|---|---|---|---|---|
| | (mg/capsule) | | (mg/capsule) | |
| Ingredients | 145 mcg | 290 mcg | 145 mcg | 290 mcg |
| MCC Spheres | 54.5 | 109 | 54.5 | 109 |
| Drug loading | | | | |
| Linaclotide | 0.145 | 0.290 | 0.145 | 0.290 |
| Hydroxy propyl methyl | 0.407 | 0.813 | 0.607 | 0.913 |
| cellulose | | | | |
| Calcium chloride | 0.975 | 1.95 | 0.875 | 2.195 |
| dihydrate | | | | |
| L-Proline | 0.422 | 0.845 | 0.622 | 1.245 |
| Hydrochloric acid | Qs | Qs | Qs | Qs |
| Purified water | Qs | Qs | Qs | Qs |
| Drug loaded pellets | 56.449 | 112.898 | 56.749 | 113.643 |
| weight | | | | |
| Seal coating | | | | |
| Hydroxy propyl methyl | 5.547 | 11.094 | 6.547 | 11.250 |
| cellulose | | | | |
| Hydrochloric acid | Qs | Qs | Qs | Qs |
| Purified water | Qs | Qs | Qs | Qs |
| Seal coated pellets | 61.996 | 123.992 | 63.296 | 124.893 |
| weight | | | | |
| Lubrication | | | | |
| Talc | 0.612 | 1.220 | 0.812 | 1.420 |
| Total pellets weight | 62.608 | 125.212 | 64.108 | 126.313 |
| Encapsulation | | | | |
| Hard Gelatin Capsule | Size 3 | Size 2 | Size 3 | Size 2 |
| Shells | | | | |

Qs—quantity sufficient

The process involved in manufacturing composition as given in Example 3 comprises the following steps:

### Drug Layering:

(i) Hydrochloric acid was added to water to get a solution of pH between 3 and 4.
(ii) Calcium chloride dihydrate and proline were added to solution of step (i) under stirring to get clear solution.
(iii) Hydroxy propyl methyl cellulose was added to solution of step (ii) and stirring continued till clear solution is obtained.
(iv) The pH of solution of step (iii) was measured and adjusted with hydrochloric acid to between 3 and 4.5.
(v) The solution of step (iv) was kept in an ice bath to attain temperature between 2 and 8° C. with nitrogen gas bubbling.
(vi) Linaclotide was added to solution of step (v) and stirring was continued for 45 minutes.
(vii) Microcrystalline cellulose beads were loaded in fluid bed coater and heated prior to drug layering.
(viii) The drug coating solution of step (vi) was sprayed on to the beads of step (vii) and dried.

### Seal Coating (Optional):

(ix) Hydrochloric acid was added to water to get a solution of pH between 3 and 5.
(x) Hydroxypropylmethyl cellulose was added to the solution of step (ix) under stirring and stirring was continued until clear solution was obtained.
(xi) Drug layered pellets were loaded in fluid bed coater followed by spraying of solution of step (x) and drying.
(xii) The pellets of step (xi) were lubricated with sifted talc and filled in capsules.

Linaclotide capsules 145 mcg of Example 1 was stored in HDPE containers with 3 g silica gel and were subjected to stability study at 40° C./75% RH.

The impurity levels in Linaclotide capsules 145 mcg at initial, 1.5 months and 3 months are given in table-1.

TABLE 1

| | 1.26 | 1.38 | 1.54 | | | Total |
|---|---|---|---|---|---|---|
| | (Oxidation | (Formaldehyde | (N-Acetyl | | | Im- |
| RRT | product) | imine product) | Linaclotide) | 1.77 | 1.81 | purities |
| Initial | 0.36 | 0.2 | 0.65 | 0.14 | 0.1 | 1.61 |
| 1.5M | 0.41 | 0.29 | 0.64 | 0.14 | 0.09 | 1.62 |
| 3M | 0.48 | 0.35 | 0.7 | 0.15 | 0.1 | 2.09 |

We claim:

1. A stable oral composition comprising linaclotide and at least one cation in an amount from about 0.5% to 5% by weight of the composition, wherein the said composition is substantially free of a primary amine.

2. The oral composition of claim 1 comprising linaclotide and at least cation selected from a group comprising Mg²+, Ca²+, Zn²+, Mn²+, K+, Na+ or Al³+ and combinations thereof, wherein said composition is substantially free of a primary amine.

3. The oral composition of claim 2, wherein at least one cation is Ca²⁺.

4. (canceled)

5. The oral composition of claim 3, wherein said Ca²+ cation is selected from the group comprising calcium chloride, calcium phosphate, or calcium sulfate and combinations thereof.

6. (canceled)

7. (canceled)

8. (canceled)

9. (canceled)

10. (canceled)

11. The composition of claim 1, further comprising one or more of pharmaceutically acceptable excipients selected from a group comprising binders, diluents, lubricants, polymers, glidants, matrix forming agents, lubricants, plasticizers and coloring agents.

12. The stable oral composition of claim 11, wherein the pharmaceutically acceptable diluent is selected from a group comprising microcrystalline cellulose, starch, mannitol, sucrose, dextrose, lactose, sorbitol, silicified microcrystalline cellulose, calcium silicate and combinations thereof.

13. (canceled)

14. The stable oral composition of claim 11, wherein the pharmaceutically acceptable binder is selected from a group comprising hydroxypropyl methylcellulose, hydroxypropyl cellulose, ethyl cellulose, polyvinyl pyrrolidone, copovidone and polyethylene glycol and combinations thereof.

15. The stable oral composition of claim 11, wherein the pharmaceutically acceptable lubricant is selected from a group comprising magnesium stearate, hydrogenated castor oil, calcium stearate, sodium stearyl fumarate, talc, vegetable oil, stearic acid and fumaric acid.

16. (canceled)

17. The stable oral composition of claim 11, wherein the plasticizer is selected from a group comprising polyethylene glycol, propylene glycol, diethyl phthalate, castor oil, triethyl citrate, tributyl citrate and dibutyl sebacate.

18. The stable oral composition of claim 11, wherein the glidant is selected from a group comprising talc, colloidal silicon dioxide, dibasic calcium phosphate, tribasic calcium phosphate and pregelatinized starch.

19. The stable oral composition of claim 1, wherein the composition is in the form of coated or uncoated granules, tablets, mini tablets, coated or uncoated beadlets filled in to hard gelatin capsules, or coated or uncoated pellets filled in to hard gelatin capsules.

20. (canceled)

21. (canceled)

22. (canceled)

23. (canceled)

24. (canceled)

25. (canceled)

26. A process for preparation of stable oral composition of linaclotide comprising:
i) loading inert cores in fluid bed processor;
ii) preparing an aqueous solution comprising a cation, hydrochloric acid, hydroxypropylmethyl cellulose and linaclotide;
iii) layering the inert cores of step (i) with the drug solution of step (ii) to form drug layered pellets;
iv) optionally seal coating the drug layered pellets using a dispersion comprising at least one polymer optionally along with one or more excipients;
v) lubricating the pellets; and
vi) encapsulating the lubricated pellets in to capsules of suitable size.

27. (canceled)

28. A method of treating a patient with irritable bowel syndrome comprising administering to a subject in need thereof the composition of claim 1.

29. The method of claim 28, wherein the irritable bowel syndrome is associated with predominant constipation and/or chronic idiopathic constipation.

30. The process of claim 26 wherein the polymer in the dispersion used in the coating step (iv) is selected from a group comprising hydroxypropylmethyl cellulose, hydroxypropyl cellulose, hydroxyethyl cellulose, polyvinyl pyrrolidone, polyvinyl alcohol and combinations thereof, optionally with one or more excipients selected from the group comprising plasticizers, glidants, diluents and combinations thereof.

31. The composition of claim 1 useful for a subject with irritable bowel syndrome.
